(19) United States
(12) Patent Application Publication
(10) Pub. No.: US 2007/0196469 A1
(43) Pub. Date: Aug. 23, 2007

(54) Magnesium microtablets with sustained release

(76) Inventor: Gunter Hopf, Tutzing (DE)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 11/497,803

(22) Filed: Aug. 2, 2006

(30) Foreign Application Priority Data
Aug. 19, 2005 (EP) 05 018 093.4

(51) Int. Cl. A61K 33/08; A61K 31/19; A61K 31/198; A61K 9/20

(52) U.S. Cl. 424/464; 424/688; 514/566; 514/574

(57) ABSTRACT

The present invention relates to magnesium coated microtablets with sustained release, i.e. magnesium slow-release products, to a regular bulk material formed therefrom, and to a bag filled with such bulk material, and to a process for producing such microtablets.

The present application claims priority under 35 U.S.C. §119 to European Patent Application No. 05 018 093.4, filed 19 Aug. 2005, a certified copy of which is filed herewith and which is hereby incorporated herein by reference as if fully set forth in its entirety.

The present invention relates to magnesium coated microtablets with sustained release, i.e. magnesium slow-release products, to a regular bulk material formed therefrom, and to a bag filled with such bulk material, and to a process for producing such microtablets.

Various magnesium compounds are on the market for the therapy of magnesium deficiency, their daily dose ranging between 100 and 400 mg of magnesium. Commercial magnesium products currently available are exclusively rapid release pharmaceutical forms such as effervescent, chewable and suckable tablets, capsules and sugar-coated tablets. Because of the rapid and complete release of active ingredient from these products, for optimal absorption of the magnesium taken the dose should be divided into three smaller amounts taken over the day. However, such a recommendation for intake usually conflicts with patient compliance. Thus, the daily dose is often taken all at once, contrary to the recommendation for intake. However, part of the dose is lost owing to the poorer absorption. This unabsorbed part of the administered dose frequently leads to side effects which are manifested in particular by diarrhoea.

In order to obtain complete absorption with a single daily intake of the total daily dose, to suppress the occurrence of unwanted side effects and, at the same time, to increase patient compliance, it is desirable to provide appropriate magnesium slow-release products. Such products ought to satisfy the requirement of a single. dose with an amount of about 15 mmol of magnesium, corresponding to 365 mg of magnesium, a sustained release over 6 to 8 hours, and the use of magnesium salts with optimal absorption properties.

In this connection, for example in the case of magnesium L-aspartate hydrochloride as active substance with high bioavailability, the relatively low magnesium content of 9.88% magnesium proves to be extremely disadvantageous. To achieve the target of a single dose, this means use of as much as 3.69 g of magnesium L-aspartate hydrochloride as active ingredient. Thus, even without further use of excipients, a resulting tablet as single dose would scarcely be possible to take, because of the size, and thus would conflict with the aim of the aforementioned optimal patient compliance.

The technical problem on which the present invention is based is to provide magnesium slow-release products or microtablets which are to be distinguished by excellent patient compliance but, at the same time, correspondingly satisfying the requirement of a single dose with an amount of about 12 to 15 mmol of magnesium.

This problem is solved by providing the embodiments characterized in the claims.

The above problem has been solved by producing coated microtablets which can be taken in a patient-friendly fashion in one dose as bulk material or regular granules.

In particular, there is provided a magnesium coated microtablet with a tablet diameter in the range from 1.6 to 2.0 mm, preferably about 1.8 to 1.9 mm, and a tablet height in the range from 2.75 to 3.10 mm, preferably 2.90 to 3.05 mm, which comprises a core comprising at least one magnesium salt, and a film coating or coating thereon, wherein the at least one magnesium salt as pharmaceutically active ingredient of the coated microtablet accounts for at least 70% by weight, preferably at least 75% by weight, of the core, and the magnesium salt is selected from magnesium oxide, magnesium carbonate, magnesium citrate, magnesium aspartate or magnesium L-aspartate hydrochloride, and the film is obtainable by applying a preferably aqueous dispersion which comprises one or more film formers, onto the core.

The magnesium salt-containing core of the coated microtablet of the invention preferably has an individual weight in the range from 8.00 to 10.00 mg, more preferably 8.25 to 9.25 mg, wherein the at least one magnesium salt as pharmaceutically active ingredient accounts for at least 70% by weight of the core. The tablet core additionally comprises at least 15% by weight of a binder selected from hypromellose, glycerol fatty acid ester such as, for example, Dynasan®, or generally customary binders for direct tabletting, such as microcrystalline celluloses, e.g. Avicel®, or directly tablettable lactose, e.g. Tablettose®. Conventional glidants and lubricants or mould release agents account for the remainder of the tablet core. An example of a glidant which can be employed is colloidal silicon dioxide (Aerosil 200). An example of a lubricant which can be used is magnesium stearate. The tablet core is the result of a direct tabletting with a magnesium active ingredient content of at least 70% by weight, preferably of at least 75% by weight, more preferably of at least 80% by weight. Such mixtures or formulations normally comprise in the prior art a maximum of only 50% active ingredient in order to achieve a satisfactory uniting of the particles and adequate flowability.

The magnesium salt as pharmaceutically active ingredient in the tablet core of the coated microtablet is selected from magnesium oxide, magnesium carbonate, magnesium citrate, magnesium aspartate or magnesium L-aspartate hydrochloride. Thus, it is also possible to employ inorganic magnesium salts such as magnesium oxide or magnesium carbonate. However, organic magnesium salts are preferred. Particularly suitable for this purpose are magnesium citrate and magnesium aspartate. Magnesium L-aspartate hydrochloride is particularly preferred.

In one embodiment of the present invention, the tablet core is composed of 75-80% by weight magnesium L-aspartate hydrochloride, 15-25% by weight hypromellose and 0.5 to 2% by weight additives selected from lubricants and glidants.

The total mass of the coated or film-coated microtablet of the invention is preferably about 8 mg to 12 mg, more preferably about 9 to 10 mg, with the total mass of the film-coated tablet preferably being adjusted depending on the magnesium salt content. The content of the coating or film coating is preferably in a range from about 1% by weight to about 15% by weight, more preferably about 2% by weight to about 10% by weight, particularly preferably about 4% by weight to about 9% by weight, based on the total mass of the coated tablet of the invention. The layer thickness of the coating or film is normally 15 to 50 μm, preferably 30 to 40 μm.

The content of the one or more film former(s) is in a range from about 60% by weight to about 90% by weight. based on the total mass of the film. The film former used according to the invention is subject to no particular restriction as long as it is pharmaceutically acceptable and suitable for slowing the diffusion of active ingredients sufficiently long, for example by swelling and forming micropores. Suitable examples are cellulose ethers such as Ethocel®, neutral (meth)acrylate-based polymers such as Eudragit® NE 30D, ionic (meth)acrylate-based polymers such as Eudragit® RS or RL or polyvinyl acetate, stabilized with PVP, such as Kollicoat® SR. In a preferred embodiment, the film former is a methacrylic acid/acrylate copolymer or a mixture of such copolymers thereof. Such film formers are obtainable under the proprietary name Eudragit®. In particular, water-insoluble, swellable film formers or coating agents based on neutral methacrylic esters and a small proportion of trimethylammoniumethyl methacrylate chloride, obtainable as Eudragit® RL or Eudragit® RS, are employed. The molar ratio of quaternary ammonium groups to the neutral ester groups is 1:20 (equivalent to about 50 meq/100 g) for Eudragit® RL, and 1:40 (equivalent to about 25 meq/100 g) for Eudragit® RS. The film-forming or coating composition additionally comprises conventional mould release agents such as, for example, glycerol monostearate and plasticizers such as, for example, triethyl citrate and, where appropriate, flavourings.

A further aspect of the present invention relates to a process for producing the above magnesium-containing coated microtablets with sustained release, comprising the steps:

(a) mixing at least one magnesium salt selected from magnesium oxide, magnesium carbonate, magnesium citrate, magnesium aspartate or magnesium L-aspartate hydrochloride, with one or more binders and conventional excipients,

(b) tabletting the resulting mixture to form tablet cores and

(c) coating the tablet cores with a coating layer, this coating being obtained by applying a preferably aqueous dispersion which comprises one or more coating agents, onto the core.

After the coating has taken place, the microtablets can be packed as regular granules or bulk material into bags or sachets or stick packs, for example with a packed weight in the range in each case from 4.0 to 6.0 g, preferably about 5.2 g. Such a bag with the regular granules from the microtablets of the invention ensures with a single dose a magnesium absorption or dose of about 10 to 20 mmol in a manner which optimally satisfies patient compliance. Based on, for example, magnesium L-aspartate hydrochloride, such a bag with a packed weight in the range in each case of about 4.0 to 6.0 g ensures with a single dose a magnesium absorption or dose of about 11.5 to 17.3 mmol. The present invention thus also relates to the use of the microtablets of the invention in the form of a regular bulk material or granules for example packed in bags or sachets or stick packs.

The following example is given in order to explain the invention in detail without restricting it.

### EXAMPLE

Production is divided into the following production steps:

Weighing of the Mixture

The following formulation was used for this purpose:

| | | Amount of single |
|---|---|---|
| | | dose |
| | Substance: | mg/microtablet |
| AI | Magnesium L-aspartate | 7.924 |
| | hydrochloride (9.88%) | |
| E | Hypromellose | 1.981 |
| E | Lubricant and glidant | 0.096 |
| | Content or amount of | |
| | magnesium/bag 15 mmol/4.67 g | |
| | Total: | 10 |

AI = Active ingredient
E = Excipient

Mixing Process

The mixing step takes place in a conventional mixer with addition of the individual components. The resulting product is free-flowing and agglomerate-free. The following IPC values were determined:

| Bulk volume | 214-220/100 g |
|---|---|
| Relative moisture | 18-21% |
| Absolute moisture | 4.25-4.34% |

Tabletting

The tabletting takes place in a commercial tablet press at a rate of approximately 50 000 tablets per hour.

The following IPC values were obtained in this case:

| | |
|---|---|
| Diameter of the tablets | 1.8 mm |
| Resistance to crushing of the tablets | 12.4-18.6 N |
| Friability (amount tested 20 g/ | not ascertainable |
| duration 10 min) | |
| Individual weights | 8.37-9.21 mg |
| Standard deviation of the individual | 1.6-2.4 |
| weights | |
| Tablet height | 2.83-2.95 mm |

The excellent tabletting properties of the mixture should be emphasized, and this with an active ingredient content of approximately 80%. The active ingredient content in commercial mixtures recommended for direct tabletting is normally 20%, and maximally 50% for optimal compression properties of the active ingredient.

This is particularly remarkable because very small tablets can be compressed with high dosage accuracy (see standard deviations) from the granules.

Coating

The hypromellose component used does not on its own provide an adequate release-slowing effect on the tablet core, owing inter alia to the very good solubility in water of the magnesium salt magnesium L-aspartate hydrochloride and its high concentration in the tablet formulation.

A coating is therefore provided to obtain sustained release. The following mechanical and technological problems must be overcome in this connection.
(i) The small diameter of the tablet core gives rise to the following problems:
   inadequate rolling properties
   high rolling weight and thus considerable attrition of the cores
(ii) The great affinity of the cores for water would imply the use of an organic coating solution. However, aqueous coating is desired and preferred.

The following automated process was therefore adapted according to the invention with the following coating formulation calculated for a batch of 180 kg of tablet cores:

| | Amount | Amount DM in |
|---|---|---|
| Substance: | kg/batch: | kg/batch: |
| Mixture of Eudragit RL 30D/ | 36.00 | 10.80 |
| Eudragit RS 30D | | |
| Mould release agent | 1.08 | 1.08 |
| Plasticizer | 2.16 | 2.16 |
| Deionized water | 16.50 | |
| Flavours | 0.42 | |
| Total amount: | 56.16 | 14.04 |

The film-formation process is noteworthy in this connection, and can be observed even after the coating. This can be speeded up by subsequent “heat treatment” at temperatures up to 40 degrees.

Packaging

After the coating has taken place, the granules are packed into bags or stickpacks with a packed weight of 5.2 g in each case.

1. A magnesium-containing coated microtablet with sustained release, which has a tablet diameter in the range from 1.6 to 2.0 mm and a tablet height in the range from 2.75 to 3.10 mm, which comprises a core comprising at least one magnesium salt, and a coating layer applied thereto, wherein the at least one. magnesium salt as pharmaceutically active ingredient of the coated microtablet accounts for at least 70% by weight of the total weight of the tablet and the magnesium salt is selected from magnesium oxide, magnesium carbonate, magnesium citrate, magnesium aspartate or magnesium L-aspartate hydrochloride.

2. The magnesium-containing microtablet according to claim 1, wherein the magnesium salt-containing core of the coated microtablet has an individual weight in the range from 8.00 to 10.00 mg.

3. The magnesium-containing microtablet according to claim 1, wherein the tablet core further comprises at least 15% by weight of a binder selected from hypromellose, glycerol fatty acid ester, microcrystalline cellulose or directly tablettable lactose, in particular hypromellose.

4. The magnesium-containing microtablet according to claim 1, wherein the tablet core is composed of 75-80% by weight magnesium L-aspartate hydrochloride, 15-25% by weight hypromellose and 0.5 to 2% by weight additives selected from lubricants and glidants.

5. The magnesium-containing microtablet according to claim 1, wherein the layer thickness of the coating is from 15 to 50 μm.

6. The magnesium-containing microtablet according to claim 1, wherein the content of the one or more coating agent(s) is in the range from about 60% by weight to about 90% by weight based on the total mass of the coating.

7. The magnesium-containing microtablet according to claim 1, wherein the coating agent is a acid/acrylate copolymer or a mixture thereof.

8. Granules formed from the magnesium-containing microtablets according to claim 1.

9. A bag filled with the granules according to claim 8 with a packed weight in the range from 4.0 to 6.0 g, so that administration of an amount of magnesium in the range from 10 to 20 mmol is achieved with a single dose.

10. A process for producing the magnesium-containing coated microtablets with sustained release according to claim 1, comprising the steps:
(a) mixing the at least one magnesium salt selected from magnesium oxide, magnesium carbonate, magnesium citrate, magnesium aspartate or magnesium L-aspartate hydrochloride, with one or more binders and conventional excipients,
(b) tabletting the resulting mixture to form tablet cores and
(c) coating the tablet cores with a coating layer, this coating being obtained by applying a preferably aqueous dispersion which comprises one or more coating agents onto the core.
